(19) United States
(12) Patent Application Publication
(10) Pub. No.: US 2026/0069882 A1
(43) Pub. Date: Mar. 12, 2026

(54) Pelvic Therapy Device with Multi-Strip Light Emission and Stimulation Modules

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN); Wen Caiyun, Shenzhen (CN); Zhou Hong, Shenzhen (CN); Chen Xin, Shenzhen (CN)

(73) Assignee: Shenzhen Kaiyan Medical Equipment Co., Ltd., Shenzhen (CN)

(21) Appl. No.: 19/324,202

(22) Filed: Sep. 10, 2025

(30) Foreign Application Priority Data
Sep. 12, 2024 (CN) 2024222556362

(51) Int. Cl. A61N 5/06; A61H 19/00; A61H 23/02

(52) U.S. Cl. CPC A61N 5/0603; A61H 19/44; A61H 23/02; A61H 2201/10; A61H 2201/5005; A61H 2205/087; A61N 2005/0611; A61N 2005/0644; A61N 2005/0651; A61N 2005/0654; A61N 2005/0659; A61N 2005/0663

(57) ABSTRACT

Embodiments of the present invention disclose a pelvic therapy device for female health applications. The device comprises an applicator body configured for insertion into a pelvic cavity, a plurality of light-emitting elements arranged to deliver therapeutic light through a light-transmissive portion of the applicator body, and a stimulation element configured to provide electrical, thermal, or mechanical stimulation to pelvic tissue. In certain embodiments, a light guide is disposed within the applicator body to direct light radially outward, and sealing structures are provided to prevent fluid ingress. The light therapy and stimulation may be applied concurrently or sequentially to enhance tissue regeneration, circulation, and neuromuscular activity. The device may further include ergonomic design features, a rechargeable power supply, and wireless control for customizable treatment. This invention offers a multimodal approach to pelvic therapy by combining phototherapy with stimulation in a compact, user-friendly form.

### TECHNICAL FIELD

The present invention relates generally to medical and wellness device technology, and more particularly to therapeutic apparatus utilizing light-based treatment. Specifically, the invention pertains to a phototherapy device configured to deliver targeted illumination, optionally in combination with mechanical stimulation such as vibration, to promote physiological benefits including pain relief, tissue recovery, and overall wellness enhancement.

### BACKGROUND

Women may experience changes in the pelvic region during childbirth or with advancing age, leading to weakened or relaxed vaginal muscles. Such conditions can result in complications, including urinary incontinence, reduced pelvic floor strength, and overall discomfort, adversely affecting daily activities and wellness.

Conventional pelvic rehabilitation devices, including exercise-based or instrument-assisted therapies, are often expensive and may have limited accessibility for regular home use. Some devices incorporate temperature sensors to monitor operating conditions and prevent tissue injury. However, sensor failure or inaccurate readings can lead to burns or other safety risks, reducing user confidence and overall device reliability.

Recent advancements have explored the use of phototherapy and mechanical stimulation, such as vibration, to promote tissue repair, improve circulation, and enhance muscle tone in the pelvic region. Nevertheless, existing light-based or vibration-assisted devices often suffer from uneven light distribution, hotspots, or inadequate mechanical coupling, which can compromise therapeutic effectiveness. Moreover, many devices are not ergonomically designed for safe and consistent contact with the vaginal canal, limiting their efficacy and comfort during use.

Accordingly, there is a need for an improved pelvic rehabilitation device that delivers uniform light therapy, optionally combined with mechanical stimulation, in a safe, ergonomic, and reliable manner to support women's pelvic health and overall wellness.

OBJECTS OF THE INVENTION

Some of the objects of the invention are as follows:

An object of the present invention is to provide a safe and effective phototherapy device for promoting women's pelvic health, particularly for strengthening or rehabilitating vaginal muscles affected by childbirth, aging, or other conditions.

Another object of the present invention is to deliver uniform light therapy along the length and circumference of the treatment section, reducing hotspots and eliminating the need for temperature sensors, thereby improving safety and reliability.

Another object of the present invention is to provide mechanical stimulation in the form of vibration, optionally in combination with light therapy, to enhance tissue recovery, blood circulation, and overall wellness.

Another object of the present invention is to offer a compact, ergonomic, and user-friendly design, including a handle and treatment section that are comfortable to hold and operate, and that ensure precise positioning of light and vibration elements for effective therapy.

Another object of the present invention is to provide a cost-effective and durable device, with simplified assembly, modular construction, and reliable sealing to prevent ingress of dust or liquids, ensuring long-term performance in home or clinical settings.

Another object of the present invention is to facilitate efficient manufacturing and maintenance, for example, through pre-grouped light strips, keyed support structures, and snap-fit assemblies that allow secure placement of components while maintaining uniform light emission and vibration delivery.

### SUMMARY OF THE INVENTION

According to a first aspect of the present invention, a pelvic therapy device is provided. The pelvic therapy device comprising: a housing having an applicator portion configured for insertion into a pelvic cavity of a female user, and a handle, the applicator portion being disposed at a predetermined angle relative to the handle; a light therapy module disposed within the applicator portion, the light therapy module comprising a light guide housing having an annular cross-section and a plurality of light strips extending axially along the light guide housing, each light strip comprising a plurality of light-emitting beads; and a light-transmissive member disposed at the applicator portion for directing the therapeutic light toward pelvic tissue; and a stimulation element disposed within the applicator portion and configured to deliver at least one of electrical, thermal, or mechanical stimulation to pelvic tissue.

In one embodiment of the invention, the predetermined angle between the applicator portion and the handle is in the range of about 140° to 170°, preferably about 158°.

In one embodiment of the invention, the applicator portion includes a distal tip having three light-emitting beads distributed circumferentially at approximately 60° intervals relative to a central axis of the applicator portion, the beads being configured to provide therapeutic irradiation to surrounding pelvic tissue.

In one embodiment of the invention, the plurality of light strips are disposed circumferentially within a single light guide housing to provide expanded irradiation area and uniform coverage of pelvic tissue.

In one embodiment of the invention, the plurality of light-emitting beads are arranged in both axial and radial directions relative to the light guide housing to achieve uniform distribution of therapeutic light and reduce localized heating.

In one embodiment of the invention, the light-emitting beads on adjacent light strips are arranged in a staggered configuration to prevent localized heat accumulation and improve homogeneity of irradiation.

In one embodiment of the invention, the applicator portion further comprises a bracket extending axially within the light guide housing, the light strips being mounted on the bracket.

In one embodiment of the invention, the bracket comprises a support arm connected to the handle and a sleeve covering the support arm, the light strips being clamped between the support arm and the sleeve.

In one embodiment of the invention, the sleeve defines a plurality of openings aligned with the light-emitting beads to permit radial emission of therapeutic light.

In one embodiment of the invention, the support arm includes opposed mounting surfaces having grooves, and the sleeve includes inward protrusions with protruding strips that engage the grooves to secure the light strips.

In one embodiment of the invention, the light guide housing comprises a first protruding ring on its outer peripheral wall, and the housing includes a second protruding ring on an inner wall, the first and second protruding rings being in sealing engagement to provide fluid resistance.

In one embodiment of the invention, the stimulation element comprises a vibration actuator disposed within the applicator portion and configured to generate vibration frequencies selectable between 10 Hz, 50 Hz, 90 Hz, and 120 Hz.

According to a second aspect of the present invention, a pelvic therapy device is provided. The pelvic therapy device comprising: a handle configured for grasping by a user; an applicator portion extending from the handle at a predetermined angle, the applicator portion including a distal tip and a light-transmissive housing; a light therapy module disposed within the applicator portion, the light therapy module comprising a plurality of light-emitting beads arranged axially and radially along the applicator portion, the distal tip including at least three light-emitting beads distributed circumferentially to provide substantially uniform irradiation of surrounding pelvic tissue; and a stimulation element disposed within the applicator portion and configured to deliver at least one of electrical, thermal, or mechanical stimulation to pelvic tissue, wherein the angled configuration of the applicator portion relative to the handle and the arrangement of the distal tip light-emitting beads are configured to provide improved contact and therapeutic coverage of the pelvic cavity.

In one embodiment of the invention, the predetermined angle between the applicator portion and the handle is in the range of about 140° to 170°, preferably about 158°.

In one embodiment of the invention, the distal tip light-emitting beads are positioned at approximately 60° intervals relative to a central axis of the applicator portion.

In one embodiment of the invention, the applicator portion further comprises a light-transmissive distal window configured to direct light emitted from the distal tip light-emitting beads toward a forward-facing portion of the pelvic tissue, thereby providing comprehensive irradiation coverage along both the axial and radial directions of the pelvic cavity.

According to a third aspect of the invention, a method of providing pelvic therapy to a female subject is provided. The method comprising: providing a pelvic therapy device comprising a housing having an applicator portion and a handle, the applicator portion being disposed at a predetermined angle relative to the handle; inserting the applicator portion into a pelvic cavity of the subject, the applicator portion comprising a plurality of light-emitting elements and a stimulation element; emitting therapeutic light from the light-emitting elements through a light-transmissive portion of the applicator portion to irradiate pelvic tissue; and actuating the stimulation element to deliver at least one of electrical, thermal, or mechanical stimulation to the pelvic tissue concurrently with or sequentially to the therapeutic light.

In one embodiment of the invention, emitting therapeutic light comprises irradiating pelvic tissue with light of a wavelength between about 600 nm and 1100 nm, including at least one of 630 nm red light, 830 nm near-infrared light, and 415 nm blue light.

In one embodiment of the invention, actuating the stimulation element comprises providing thermal stimulation to raise or lower temperature of the pelvic tissue by at least 2-5°C.

In one embodiment of the invention, the therapeutic light and the stimulation element are operated in alternating cycles of light emission and stimulation delivery.

In the context of this specification, when an element is described as being “fixed to,” “disposed on,” or “secured to” another element, such terminology shall be interpreted to include both direct and indirect connections, unless expressly stated otherwise. Similarly, when an element is described as being “connected to” or “coupled with” another element, such terminology may include direct or indirect mechanical, electrical, optical, or thermal connections.

In the context of this specification, the terms “first,” “second,” “third,” and the like are used solely for descriptive purposes to distinguish features or components and do not imply priority, sequence, relative importance, or limitation on the number of such features, unless otherwise expressly stated.

In the context of this specification, the term “plurality” means two or more, unless otherwise expressly indicated. Similarly, the term “several” refers to more than one, unless expressly limited in scope.

In the context of this specification, the term “LED” refers to one or more light-emitting diode (LED) elements or modules, configured to emit light within therapeutic wavelength ranges. The LED module may further include associated driver circuitry, thermal management components (e.g., heat sinks, conductive substrates), and optical structures (e.g., reflectors, diffusers, or lenses) to control light emission and distribution within the pelvic treatment zone.

In the context of this specification, the terms “light,” “irradiation,” “illumination,” and “emission” refer to electromagnetic radiation spanning visible to infrared wavelengths, specifically optimized for photobiomodulation and therapeutic applications within pelvic tissues. Preferably, the emitted light is low-level red and near-infrared (NIR) light. Infrared radiation as used herein includes Near IR (750-1400 nm), Short-Wavelength IR (1400-3000 nm), Mid-Wavelength IR (3000-8000 nm), Long-Wavelength IR (8000-15,000 nm), and Far IR (15,000-1,000,000 nm). The therapeutic device operates at low energy densities, typically below about 500 mW/cm², to promote tissue healing, enhance circulation, and reduce inflammation, without causing ablation, cutting, or thermal coagulation of tissues.

In the context of this specification, the term “light source” or “phototherapy source” refers to any suitable light-emitting element, including LEDs, Superluminous Diodes (SLDs), Organic Light-Emitting Diodes (OLEDs), or other optoelectronic devices, configured to generate therapeutic wavelengths.

In the context of this specification, the term “lamp board” refers to a printed circuit board (PCB), flexible strip, or substrate upon which a plurality of light sources are mounted and electrically interconnected with the control circuitry and power supply contained within the handle portion of the device.

In the context of this specification, the term “light guide housing” refers to a transparent or translucent tubular structure configured to enclose, support, and guide light emitted by the light sources toward the pelvic tissue. The housing may further incorporate features such as ribs, grooves, or snap-fit joints for alignment, sealing, and assembly.

In the context of this specification, the term “control switch” or “button” refers to a mechanical, capacitive, or electronic actuator disposed on the surface of the handle, configured to control activation, mode switching, brightness adjustment, or vibration intensity of the device.

In the context of this specification, “vibration element” or “vibrator” refers to a mechanical or electromechanical component (such as an eccentric rotating mass motor or linear resonant actuator) disposed within the device, configured to generate vibratory stimulation. Such stimulation is intended to enhance circulation, facilitate muscular relaxation, and improve therapeutic outcomes when combined with phototherapy.

In the context of this specification, “waterproofing” or “watertight seal” refers to structural arrangements, such as sealing rings, raised ridges, interlocking joints, or compression fittings, configured to prevent ingress of bodily fluids, cleaning solutions, or external contaminants into the handle or electronic compartments of the device.

In the context of this specification, “light-emitting diodes (LEDs)” shall include, but not be limited to, high-power LEDs, surface-mounted LEDs, flexible strip LEDs, and wavelength-specific therapeutic LEDs. LEDs may be configured to emit in red (e.g., 630-680 nm), near-infrared (e.g., 800-900 nm), or other bands such as yellow or green for tissue-specific therapeutic purposes.

Materials used in the fabrication of the LEDs may include semiconductor compounds such as Gallium Nitride (GaN), Indium Gallium Nitride (InGaN), Aluminum Gallium Indium Phosphide (AlGaInP), or other III-V group semiconductors. Different dopants or phosphor coatings may be employed to achieve desired emission wavelengths and therapeutic characteristics.

In addition to conventional inorganic LEDs, one or more embodiments may incorporate organic light-emitting diode (OLED) panels or flexible inorganic LED sheets, adapted for conformability within the pelvic application region. These may provide broader light distribution, improved comfort, and reduced rigidity compared to conventional rigid lamp boards.

In the context of this specification, the term “stimulation element” may further encompass electrodes configured to provide complementary therapies, such as Transcutaneous Electrical Nerve Stimulation (TENS), Electrical Muscle Stimulation (EMS), or Microcurrent Therapy (MET), applied in conjunction with light therapy to enhance pelvic muscle rehabilitation and pain relief.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a light therapy device in accordance with an embodiment of the present invention.

FIG. 2 illustrates a schematic structural view of the light therapy device of FIG. 1, shown in a state removed from the light guide housing.

FIG. 3 illustrates an exploded view of the light therapy device of FIG. 1, depicting the relative arrangement of its internal components.

FIG. 4 illustrates a cross-sectional view of the light therapy device of FIG. 1, showing the internal configuration of the light source and light guide housing.

FIG. 5 illustrates a partial exploded view of the light therapy device of FIG. 2, highlighting the connection of multiple light strips.

FIG. 6 illustrates a partial cross-sectional view of the light therapy device of FIG. 2, further detailing the structural relationship of the light strips within the housing.

FIG. 7 is a side view of a light therapy device illustrating the dimensional proportions of the handle portion and applicator portion, including the curvature of the applicator portion relative to the handle, and showing the arrangement of multiple light-emitting elements along the length of the applicator portion.

FIG. 8 illustrates the distal end of the light therapy device, showing a set of three light-emitting diodes (LEDs) strategically positioned.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practised. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention disclose a phototherapy device for promoting women's pelvic health, particularly for strengthening or rehabilitating vaginal muscles that may weaken or relax due to childbirth, aging, or other physiological conditions. The device is designed to deliver targeted therapeutic light, optionally combined with mechanical stimulation, in a safe, ergonomic, and user-friendly manner. The device may be used in clinical settings or for home-based therapy, offering an effective, low-risk, and cost-efficient alternative to conventional rehabilitation methods.

The device generally comprises a handle and a treatment section. The handle is configured for comfortable manual operation and houses a battery and a circuit board electrically connected to the battery. The handle may include anti-slip surfaces such as grooves, bumps, or textured coatings to enhance grip and prevent accidental slippage during therapy. In an embodiment, the handle is formed of a durable, lightweight material, and its color and material may differ from the treatment section to provide a visually distinct and ergonomic design.

The treatment section includes a light guide housing and multiple light strips disposed within the housing and electrically connected to the circuit board. Each light strip comprises a plurality of light beads spaced apart along the strip, which may include one or more of red, blue, or infrared LEDs. These light beads are configured to emit light sufficient to stimulate tissue repair, enhance blood circulation, and promote muscle recovery in the pelvic region. The light guide housing may have a circular, oval, or otherwise ergonomically shaped cross-section and is preferably made from medical-grade silicone or other soft, biocompatible materials to provide comfort and safe contact with tissue.

The light strips extend axially along the treatment section and are arranged circumferentially around the central axis of the light guide housing. In one embodiment, the light beads on adjacent light strips are staggered to ensure uniform light distribution in both axial and radial directions. This configuration reduces heat concentration at any single location and eliminates the need for conventional temperature sensors, thereby increasing safety and reliability. The treatment section may include flared or angled openings for the light beads, which improve the light emission angle and expand the treatment area within the vaginal canal.

In certain embodiments, the treatment section further comprises a bracket inserted within the light guide housing. The bracket may include a support arm and a surrounding housing to which the light strips are mounted. The support arm may be generally tubular or otherwise shaped for structural rigidity and may contain one or more vibrators electrically connected to the circuit board. The housing may include openings aligned with the light beads to facilitate light emission. The inner surfaces of the housing may be polygonal, forming grooves, ridges, or keyed surfaces to secure the light strips in precise positions, preventing twisting or deformation during use. Multiple light strips may be pre-grouped and connected end-to-end to simplify assembly, ensure uniform light distribution, and reduce manufacturing complexity.

The device may incorporate one or more vibrators disposed within the support arm to provide mechanical stimulation. The vibration module operates only when the light assembly is active, ensuring controlled and safe operation. In various embodiments, the vibration module may offer multiple frequencies, such as 20 Hz, 50 Hz, 90 Hz, and 120 Hz, allowing users to select low, medium, or high-intensity vibration modes. The combination of light and vibration therapy may improve tissue perfusion, stimulate muscle contraction, and enhance the overall effectiveness of pelvic rehabilitation.

The handle may include control buttons or a touch-sensitive interface to allow users to: Power the device on or off; Adjust light intensity or brightness; Select light wavelength modes (e.g., red, blue, or infrared); and Adjust vibration frequency and intensity.

The buttons may provide tactile, visual, or haptic feedback, and may be configured with different press durations for different functions. For instance, a long press may power the device on or off, while short presses may cycle through vibration modes or light intensity levels.

The device is designed with multiple safety features. Uniform light emission, achieved through staggered LEDs and flared emission openings, minimizes hotspots and eliminates the need for temperature sensors, reducing the risk of burns. The light guide housing is made of soft, biocompatible material to provide safe tissue contact. The support arm and housing are constructed from durable, elastic plastic to maintain alignment and positioning of the light strips and vibration modules. Snap-fit, interlocking, and waterproof sealing structures secure the light guide housing to the handle, preventing ingress of water, dust, or other contaminants.

In one embodiment, the phototherapy device is configured to provide adaptive and personalized therapy through the integration of optical, pressure, and/or proximity sensors. These sensors continuously monitor tissue response and dynamically adjust the light intensity, wavelength, and vibration frequency to deliver optimized therapy based on the user's specific physiological condition. Such adaptive control ensures safe, effective, and individualized treatment.

The device further incorporates thermal modulation elements, including gentle heating and cooling components, which operate in conjunction with the light and vibration therapy to enhance blood circulation, improve tissue repair, and provide additional comfort during use.

In an embodiment, the light therapy device further comprises a stimulation element configured to provide supplementary therapeutic effects in conjunction with phototherapy. The stimulation element may be selected from, or include combinations of, an electrical stimulation module, a thermal stimulation module, or a mechanical stimulation module. For example, the electrical stimulation module may be disposed adjacent to or integrated with the light guide housing, and may deliver low-level microcurrent or neuromuscular stimulation through conductive contact surfaces arranged along the applicator portion of the device. Such stimulation assists in promoting localized blood circulation, enhancing muscle relaxation, and supporting tissue regeneration within the pelvic region.

In other embodiments, the stimulation element may comprise a thermal regulation unit, such as a resistive heater or Peltier-based module, that provides mild heating or cooling cycles. This feature can be synchronized with the light therapy cycle, thereby amplifying therapeutic outcomes by soothing discomfort, reducing inflammation, or improving user comfort during treatment.

The light-emitting assembly comprises a plurality of LEDs capable of emitting multiple wavelengths, including but not limited to red, blue, infrared, yellow, and green light. The combination of these wavelengths targets specific tissue repair pathways, promotes anti-inflammatory effects, stimulates collagen and elastic tissue formation, and improves overall pelvic wellness.

The light and vibration outputs are configurable to operate in pulsed or modulated patterns, delivering intermittent stimulation or patterned sequences to mimic professional therapy protocols and enhance tissue recovery. Such pulsed or modulated operation can be applied individually to light, vibration, or both simultaneously.

The treatment section is modular, with removable, sterilizable, or size-customizable heads designed to accommodate anatomical variations among users. The modular design facilitates maintenance, cleaning, and replacement of components without compromising therapeutic efficacy.

The device includes connectivity and data logging features, such as Bluetooth or Wi-Fi modules, enabling integration with mobile applications. These systems allow users to track therapy sessions, monitor progress over time, receive reminders, and access cloud-based analytics for personalized treatment optimization.

To provide real-time feedback to the user, the device is equipped with visual and auditory indicators, including LEDs, symbols, or gentle alerts, indicating therapy status, selected light intensity, vibration mode, or session completion.

The ergonomic design of the device includes soft-touch handles, adjustable curvature of the light guide housing, and biocompatible materials with antibacterial or hygienic coatings, ensuring comfort, hygiene, and ease of use. The housing and handle are designed to conform to the natural contours of the pelvic region, further improving user experience and therapeutic efficiency.

The device is configured to allow modular upgrades, including replaceable light strips, vibration modules, or sensor components. Such modularity ensures long-term durability, facilitates the incorporation of newer technologies, and enables expansion of therapeutic functionality without replacement of the entire device.

Together, these features provide a phototherapy device that delivers safe, effective, and personalized pelvic health therapy, integrating light, mechanical stimulation, thermal modulation, adaptive feedback, and user-centered design to address the unique needs of each user.

In operation, the user holds the handle and positions the treatment section appropriately within the pelvic region. Upon activation, the light beads emit therapeutic light, optionally accompanied by vibration, to stimulate tissue repair, improve blood circulation, and strengthen pelvic muscles. The device may be operated at various brightness levels, light wavelengths, and vibration frequencies to suit the user's comfort and therapeutic needs. The ergonomic design, coupled with safety interlocks and modular construction, ensures that therapy is delivered effectively and reliably while minimizing risks such as burns or discomfort.

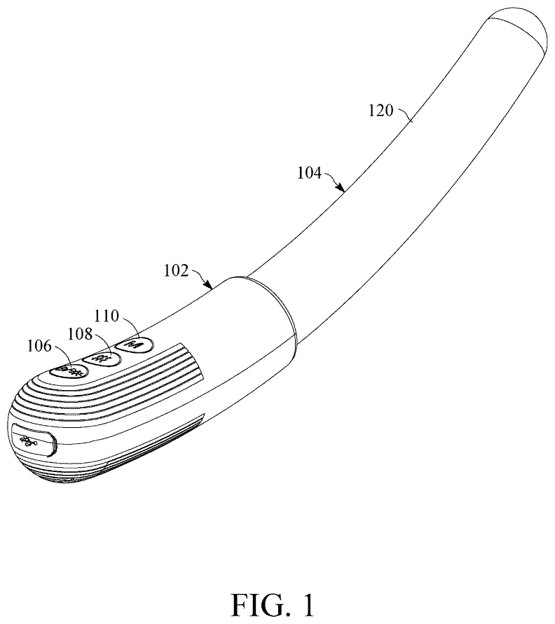

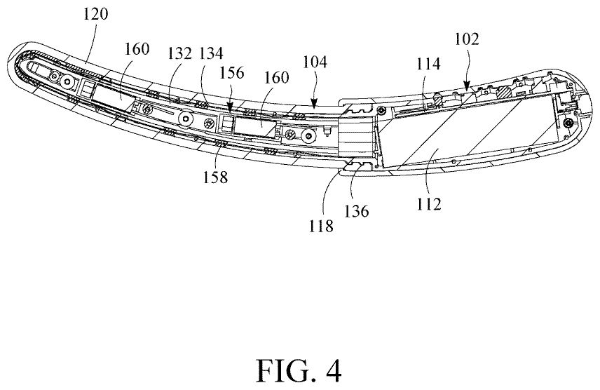

Embodiments of the present invention are now described in conjunction with FIGS. 1 to 8, for the purpose of illustrating the structure, components, and operation of the device

Referring to FIGS. 1 to 3, the present invention provides a phototherapy device for promoting pelvic wellness in women. The device generally comprises a handle 102 and a treatment section 104, which is connected to form a handheld, ergonomic unit suitable for user operation.

The handle 102 houses a battery 112 and a circuit board 114 electrically connected to the battery 112, providing the power and control circuitry required for operating the light-emitting and vibration modules of the device. The handle 102 is configured for secure and comfortable handholding, with its outer surface including an anti-slip structure, which may include grooves, bumps, ridges, or combinations thereof, to prevent slippage during use. The handle and treatment section may have different material compositions or colors to improve visual distinction, ergonomic recognition, or aesthetic appeal, with the handle optionally formed in light gray. The connection between the handle 102 and the treatment section 104 is designed to be waterproof, meeting standards such as IPX6 or higher, thereby ensuring safety and reliability during cleaning or exposure to fluids.

In some embodiments, the pelvic therapy device includes a USB charging interface positioned at the handle portion. To achieve both functional reliability and user comfort, the USB cover 116 is formed entirely of a silicone material. The cover design is particularly challenging due to the restricted spatial constraints in this region of the device, which limit the available volume for the connector while requiring a seamless exterior profile. The USB cover 116 is therefore configured such that the underlying plastic components of the connector are concealed, thereby maintaining a hygienic, smooth, and aesthetically pleasing surface.

A further design consideration is the requirement for liquid ingress protection. The silicone cover is structured to provide a sealing effect sufficient to achieve at least an IPX6 level of waterproofing, ensuring safe operation during cleaning and therapeutic use. This may be realized through precision molding techniques, integration of sealing rings, or overmolding of silicone directly around the connector region. These measures allow the device to maintain robust waterproofing performance, while also enabling feasible mass production assembly despite the limited design space.

The treatment section 104 comprises a light guide housing 120 and multiple light strips 156 arranged within the housing and electrically connected to the circuit board 114. The light assembly 130 consists of these multiple light strips 156, each of which may be formed on a flexible substrate to allow for slight curvature or deformation during assembly or use. Each light strip 156 carries a plurality of light beads 158 spaced along its length. The light guide housing 120 is generally circular in cross-section and may be made of transparent or medical-grade silicone, providing both optical clarity for light transmission and soft contact for user comfort.

Each light strip 156 extends axially along the length of the light guide housing 120 and is arranged circumferentially around the centerline of the light guide housing 120. This configuration allows the light beads 158 to be distributed evenly along both the axial and radial directions of the treatment section 104, thereby ensuring uniform illumination and minimizing localized heat accumulation. The even distribution of light prevents the need for separate temperature sensors, reducing manufacturing costs and enhancing safety by avoiding potential burns due to sensor failure.

In a particular embodiment, the light beads 158 on two adjacent light strips 156 are staggered. This staggered arrangement further improves uniformity of light emission, disperses heat effectively, and reduces the likelihood of concentrated thermal spots on tissue. The light beads 158 on each light strip 156 may be equally spaced, such that the spacing between adjacent beads on one strip complements the positions on neighboring strips, forming a regular staggered pattern. This design ensures consistent therapeutic exposure throughout the treatment session, promoting stimulation of tissue repair, collagen formation, and muscle enhancement in the pelvic region.

The light therapy device provided in this application features multiple light strips 156 within the light guide housing 120 of the treatment section 104. The light emitted by these light strips 156 improves the corresponding muscle tissue. Each light strip 156 extends axially and surrounds the centreline of the light guide housing 120. Multiple light beads 158 are spaced apart along the length of the light strips 156, ensuring that the light beads 158 are distributed in both the axial and radial directions of the treatment section 104.

This results in more uniform light emission and less heat concentration, eliminating the need for a temperature sensor, saving costs, and preventing burns caused by temperature sensor failure. This ensures safety and reliability. In one embodiment, the light beads 158 on two adjacent light strips 156 are staggered. This ensures a more even distribution of the light beads 158 and disperses the heat, thus preventing burns during use.

The light beads 158 on the light strips 156 can be arranged evenly spaced, with the spacing between adjacent light beads 158 on each light strip 156 being equal. This allows the light beads 158 on adjacent light strips 156 to form a relatively regular staggered arrangement.

In certain embodiments, the treatment section of the applicator portion comprises six groups of light strips, each group incorporating a plurality of lamp beads. The layout of these lamp beads is carefully designed to balance therapeutic effectiveness with manufacturing feasibility. From a therapeutic perspective, the relative number and positions of the lamp beads are selected to achieve uniform irradiation of pelvic tissue. In some implementations, the lamp beads are arranged in a staggered configuration across adjacent strips, which minimizes localized heat accumulation and prevents the formation of “hot spots.” Alternatively, the lamp beads may be disposed in a matrix arrangement that provides consistent spacing in both axial and radial directions. Both configurations ensure that the emitted light is distributed more evenly throughout the treatment area, thereby enhancing therapeutic outcomes.

From a structural and manufacturing perspective, the arrangement also takes into account the complexity of mold fabrication and production assembly. By optimizing the number and placement of lamp beads, the design reduces unnecessary variations in strip geometry and facilitates efficient assembly of the light guide housing with the embedded lamp strips. This balanced design consideration provides both improved clinical performance and ease of manufacturability, enabling scalable production without compromising on safety or treatment quality.

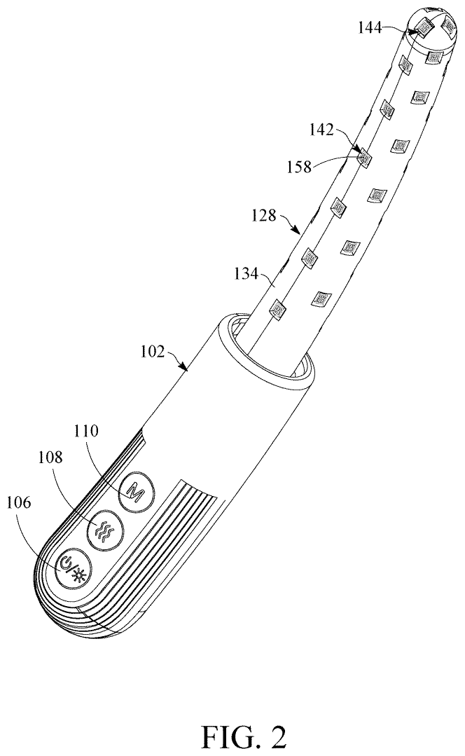

Referring to FIGS. 2 to 4, the treatment section 104 further includes a bracket 128, which is inserted into the light guide housing 120 and extends longitudinally along the housing. The bracket 128 provides a mounting and positioning structure for multiple light strips 156, which are fixed to the bracket at circumferential intervals around the centerline of the light guide housing 120. The bracket 128 may be formed from a durable plastic material and has one end inserted into and fixedly connected to the handle 102, ensuring stable mechanical and electrical integration between the handle and the treatment section.

The bracket 128 comprises a support arm 132 and a sleeve 134 that fits over the support arm 132 to form an enclosure for the light strips 156 and other components. The sleeve 134 can be either an integral unit or split into two separate parts; in this embodiment, it is a split housing consisting of two plastic strips assembled using screws. One end of the sleeve 134 is inserted into the handle 102 and fixedly connected thereto, with close contact against the inner wall of the handle, providing a secure and waterproof junction.

The sleeve 134 includes first through-holes 142 positioned to align with each light bead 158, allowing light emission from the light strips. Additionally, second through-holes 144 are formed at the end of the housing distal from the handle 102, through which some of the light beads 158 extend, increasing the area of light emission and thereby enlarging the tissue treatment zone. Both the first through-holes 142 and the second through-holes 144 may be flared, allowing the light beads 158 to emit light over a wider angle, improving coverage and therapeutic effect.

In the illustrated embodiment, the light assembly 130 comprises six light strips 156, with seven light beads 158 spaced along each of three non-adjacent strips, and six light beads 158 spaced along the other three non-adjacent strips. The end surface of the sleeve 134 distal from the handle is curved, and the second through-holes 144 are positioned within this curved surface, enabling light from selected light beads 158 to be emitted from the distal portion of the treatment section.

The support arm 132 is generally tubular, providing structural support while housing internal components. It has an opening at the end near the handle 102 for assembly and connection purposes. The support arm 132 can be formed by joining two plastic shells using screws, providing a durable and precisely aligned enclosure.

At least one vibrator 160 is housed within the support arm 132 to provide mechanical stimulation in combination with light therapy. In this embodiment, two vibrators 160 are spaced along the support arm 132, one near the handle end and the other near the distal end. Activation of the vibrators is controlled via a function button, allowing simultaneous operation at the same or different frequencies, or deactivation as desired.

The light beads 158 can include red, blue, and infrared LEDs, allowing the device to provide multi-wavelength phototherapy. Red and blue light may be used for skin-related effects such as wrinkle reduction, pain relief, and anti-inflammation, while infrared light promotes blood circulation and tissue metabolism.

Both the sleeve 134 and the support arm 132 are formed from elastic plastic, enabling the light strips 156 to be elastically clamped between the housing and support arm, securing the strips while allowing slight flexibility for assembly and use. Light emitted by the light beads 158 passes through the light guide housing 120 and is emitted outward, providing uniform illumination across the treatment area.

The sleeve 134 and support arm 132 are elongated cylindrical structures, and the radial cross-section of the inner wall of the sleeve 134 is polygonal, with each side dimensioned to match the width of the respective light strip 156. This polygonal inner structure provides precise positioning and prevents twisting or deformation of the light strips, ensuring consistent light output and reliable operation of the device.

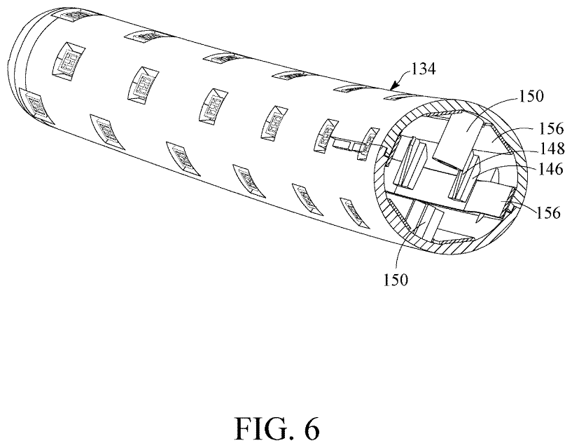

As illustrated in FIG. 6, the radial cross-section of the inner wall of the sleeve 134 is configured as a dodecagonal structure, comprising twelve flat sides arranged around the central axis. Each side of the dodecagon is dimensioned to correspond to the width of a respective light strip 156, with flat surfaces separating adjacent light strips. This geometric configuration ensures that each light strip 156 is securely positioned, preventing twisting, lateral displacement, or deformation that may occur due to compression during assembly or operation.

The polygonal cross-section functions as an integrated positioning and installation structure, providing precise alignment for the light strips 156 without requiring additional fasteners or alignment tools. By fitting the light strips 156 within the sides of the dodecagonal inner wall, the assembly process is simplified, enabling quick and accurate installation of multiple light strips simultaneously. Moreover, the structure maintains consistent spacing and orientation of the light strips 156, ensuring uniform light emission and optimal therapeutic coverage across the treatment section.

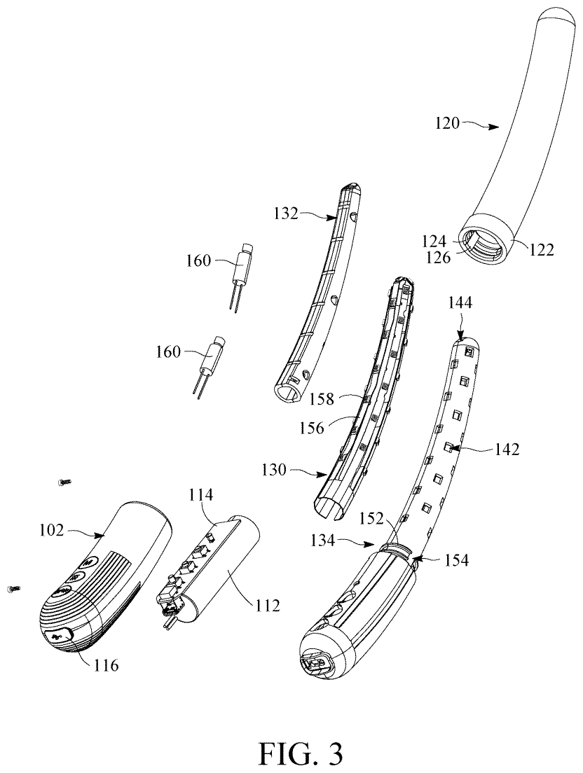

Referring to FIGS. 3 and 6, the plurality of light strips 156 is organized into at least two groups, each group comprising a minimum of two light strips 156 whose ends are mechanically or electrically connected. Within each group, the connected arrangement allows the light strips 156 to be handled as a single unit during assembly. This configuration eliminates the need to insert each light strip individually into the sleeve 134, thereby significantly improving the efficiency and speed of the assembly process.

By grouping the light strips 156, the device ensures consistent alignment and spacing of the strips within the polygonal inner wall of the sleeve 134, reducing the risk of misalignment or twisting during installation. Additionally, this grouping facilitates simultaneous placement of multiple light strips, streamlining manufacturing and reducing labor time, while maintaining uniform light distribution and mechanical stability throughout the treatment section.

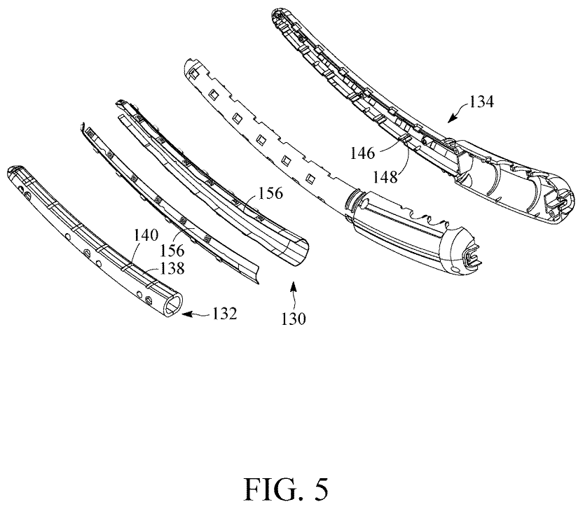

As illustrated in FIGS. 5 and 6, the light assembly 130 comprises two distinct sets of light strips 156, one set including two light strips and the other set including four light strips. Within each set, the ends of the light strips 156 near the handle 102 are interconnected, allowing the strips to be handled as a single unit during assembly. This interconnected arrangement facilitates simultaneous insertion of multiple light strips 156 into the sleeve 134, improving assembly efficiency and ensuring consistent alignment.

To precisely position and secure the light strips 156, coordinating positioning structures are provided on both the inner wall of the sleeve 134 and the outer wall of the support arm 132. Mounting surfaces 138 for the light strips are provided on opposing sides of the support arm 132 and are separated by grooves 140. On the inner wall of the sleeve 134, corresponding protrusions 146 and ridges 148 engage with the grooves 140 on the support arm 132, forming a secure and stable clamping structure. The gap between each protrusion 146 and the adjacent inner wall forms a slot for edge engagement of the light strips 156, preventing lateral movement or twisting.

Additional positioning posts 150, such as cross-shaped or hollow posts, are provided on the inner wall of the sleeve 134 and align with matching through-holes in the support arm 132, thereby securing the sleeve 134 and support arm 132 together. One end of the sleeve 134 extends into the handle 102 and is fixed thereto, aligning with the contour of the handle's inner wall and secured with screws. The light guide housing 120 similarly extends into the handle 102 and seals against the sleeve 134, forming a watertight connection that prevents ingress of liquids or dust into the handle or treatment section 104.

At one end of the light guide housing 120, specifically the axially open end that interfaces with the handle 102, a first raised ring 122 is formed on the outer circumference. This raised ring projects radially outward and provides a defined contact surface for engaging with the handle. Correspondingly, a second raised ring 118 is radially projected from the inner wall of the handle 102, near the opening of the treatment section 104. The second raised ring is positioned such that, upon insertion of the light guide housing 120 into the handle 102, the second ring abuts the first ring, creating a tight sealing contact that resists ingress of liquids, dust, or other external contaminants. This configuration ensures that the interface between the handle and the treatment section maintains waterproof integrity, meeting or exceeding predetermined IPX6 standards or similar requirements.

One end of the sleeve 134, which forms part of the support bracket of the light assembly 130, extends into the handle 102 and is fixedly secured thereto, providing additional structural support. The portion of the light guide housing 120 that extends into the handle is interlocked with the sleeve 134 via a mating interlocking structure 136. This interlocking structure comprises complementary projections, grooves, or snap-fit features on the light guide housing 120 and the sleeve 134, ensuring that the two components are mechanically secured in both axial and radial directions.

The combination of the raised rings (122, 118) and the mating interlocking structure 136 provides a redundant sealing mechanism, enhancing the waterproof performance of the device and ensuring that the treatment section 104 remains sealed even under external pressure, vibration, or handling during use. This arrangement not only prevents leakage of liquids into the handle 102 and onto the electronic components, including the battery 112 and circuit board 114, but also contributes to the overall mechanical stability of the light assembly, minimizing movement or misalignment of the light guide housing 120 during therapy.

To further enhance positioning stability, the inner wall of the light guide housing 120 at the handle-facing opening includes multiple annular first ribs 124, which are discontinuous and separated by positioning blocks 126. The housing 134 has corresponding second ribs 152 with break points forming grooves 154 that receive the positioning blocks 126. When the light guide housing 120 is inserted, the first ribs 124 snap into the grooves formed by adjacent second ribs 152, and vice versa, creating a stable snap-fit connection that maintains alignment and mechanical stability.

The handle 102 is equipped with three function buttons: a first button 106, a second button 108, and a third button 110, which may be spaced 5 -25 degrees apart along the same side of the handle. The first button 106 serves dual functions: a long press powers the device on or off, while a short press adjusts the vibration intensity of the vibrator 160, enabling multiple vibration levels. The second button 108 adjusts the brightness of the light assembly 130, allowing selection of two or more brightness settings. The third button 110 switches the light between different wavelength modes, such as red, blue, or infrared, to provide therapy targeted to specific tissue responses.

The vibrator 160 is activated only when the light assembly 130 is operational and cannot be activated independently, ensuring coordinated operation of light and vibration therapy. The device does not have a separate massage function; instead, vibration is integrated with light therapy for safe and effective pelvic treatment.

This configuration ensures ergonomic handling, precise positioning of light strips, waterproof integrity, and coordinated multi-modal therapy, enhancing the safety, reliability, and therapeutic efficacy of the device.

As shown in FIGS. 1 to 3, the handle 102 of the light therapy device includes three function buttons, namely a first button 106, a second button 108, and a third button 110, which are arranged in a straight line along the top surface of the handle for convenient thumb or finger access during use. Each button is provided with a corresponding graphic, symbol, or text marking on its surface, indicating the function associated with the button and whether the function is currently active or inactive, thereby improving user interface clarity and operability.

The first button 106 serves a dual function, combining power control and vibration level adjustment into a single user interface element. Specifically, a long press of the first button 106, lasting approximately 1.5 to 2 seconds, powers on the device, while a prolonged press of approximately 2.5 seconds or longer powers off the device. A short press of the first button 106, typically in the range of 0.2 to 0.5 seconds, is used to control the vibrator 160. Upon the first short press, the vibrator 160 activates at the default vibration frequency of 20 Hz. Subsequent short presses sequentially cycle the vibration frequency through 50 Hz, 90 Hz, and 120 Hz, with a further short press deactivating the vibrator. This configuration allows the user to select from four vibration intensity modes, providing flexibility for low-intensity, medium-intensity, high-intensity, and maximum-intensity massage therapy.

In alternative embodiments, the vibrator 160 may operate within a reduced range of 20 Hz to 90 Hz, providing three vibration levels corresponding to 20 Hz, 50 Hz, and 90 Hz. This allows users to perform tailored pelvic or tissue massage therapy, adjusting the intensity according to comfort, therapeutic need, or sensitivity of the tissue.

The second button 108 is dedicated to adjusting the brightness of the light emitted by the light assembly 130. In the illustrated embodiment, the light assembly can be set to two discrete brightness levels, specifically 50% and 100%. Upon activation at 100% brightness, pressing the second button 108 reduces the illumination to 50%, and a subsequent press returns the brightness to 100%. This two-level brightness control allows the user to adapt light intensity to comfort and therapy requirements.

The third button 110 allows the user to switch the light emission between different wavelength modes, thereby selecting different combinations of red, blue, or infrared light as desired for specific therapeutic effects, such as tissue repair, blood circulation enhancement, anti-inflammation, or pain relief.

Notably, the vibrator 160 is interlocked with the light assembly 130, such that the vibration function cannot operate independently of the light therapy function. This integration ensures that the massage effect is applied in conjunction with phototherapy, providing a synergistic therapeutic effect. Collectively, this button arrangement and control strategy enable ergonomic, intuitive, and precise adjustment of both light and vibration therapy functions, improving safety, user experience, and therapeutic efficacy.

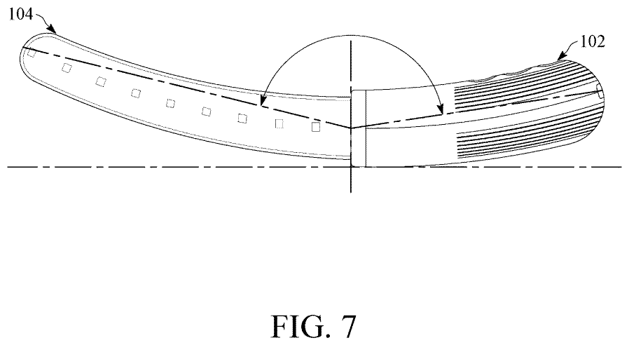

Referring to the FIG. 7, the pelvic therapy device comprises the handle portion 102 and the treatment or applicator portion 104 extending forwardly from the handle 102. The applicator portion 104 is configured for insertion into a pelvic cavity and is shaped with a curved profile, defining an included angle in range of 140 degree to 170 degree between the central longitudinal axes of the handle 102 and the applicator portion 104. In a preferred embodiment, the angle between the central longitudinal axes of the handle 102 and the applicator portion 104 is preferably 158 degree. This curvature is specifically designed to follow the natural anatomical orientation of the female pelvic cavity, thereby enabling improved contact with the target tissue and enhancing therapeutic efficiency while maintaining user comfort.

The applicator portion 104 has an overall length of about 132.7 mm measured from the junction with the handle 102 to the distal tip, and a maximum width of about 47.0 mm. The handle portion 102 extends approximately 105 mm in length and has a reduced height dimension of about 29.9 mm, which allows the handle 102 to be conveniently grasped by the user or clinician. The ergonomic shaping of the handle 102, which includes surface grooves or ridges, further improves grip stability during use.

Within the applicator portion 104, a plurality of light-emitting beads (for example, LEDs) are arranged in a distributed manner along the axial length and across the radial surface of the applicator. The arrangement of the light beads ensures that therapeutic light is directed uniformly over a large area of pelvic tissue. The spacing of the light beads is selected to minimize localized thermal accumulation, thereby avoiding discomfort or tissue damage. The beads may be staggered along adjacent rows to enhance uniformity of irradiation and reduce the risk of hot spots.

The applicator portion 104 includes a light-transmissive housing or window through which the therapeutic light is emitted. The cross-sectional geometry is contoured to match pelvic anatomy, while also maintaining sufficient internal volume to accommodate the light strips, wiring, and optional stimulation modules. The measured curvature and dimensional proportions of the applicator portion are optimized to reach target zones of the pelvic floor while permitting ease of insertion and withdrawal.

The geometric design (length, width, angle, curvature) directly contributes to both the therapeutic coverage and the user's comfort. The ergonomic relationship between the handle and applicator portion provides stability, precision, and safety in delivering combined light therapy and stimulation treatments within the pelvic cavity.

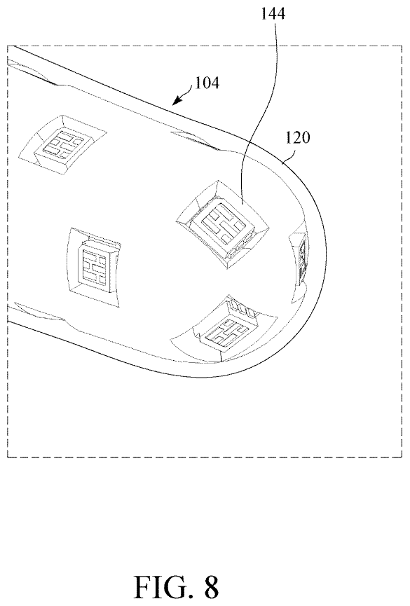

FIG. 8 illustrates the distal end (treatment tip) of the pelvic therapy device, wherein a set of three light-emitting diodes (LEDs) 144 are strategically positioned. These LEDs 144 are arranged at 60° intervals around the central longitudinal axis of the applicator tip, thereby forming a circumferentially uniform distribution. This specific angular arrangement ensures that the emitted therapeutic light covers not only the lateral surfaces of the pelvic cavity but also the forward-facing end region of the applicator.

The positioning of the three LEDs on the top surface of the treatment section allows the distal end of the pelvic wand to deliver light energy directly to tissue regions that may otherwise receive less exposure from axially aligned LEDs along the shaft. By providing illumination from both the shaft and the tip, the device achieves comprehensive irradiation coverage, enhancing therapeutic effectiveness.

Furthermore, the angled configuration reduces shadowing and hot-spot formation, enabling a more homogeneous light distribution at the distal tip. This arrangement also complements the axial and radial light-emitting beads disposed along the length of the applicator, thereby extending the therapeutic zone into the deepest insertion region.

The inclusion of distal tip LEDs makes the device particularly effective for conditions requiring targeted treatment at the apex of the vaginal canal, improving clinical outcomes compared to conventional devices lacking forward-directed irradiation.

In an embodiment, the light therapy device further comprises a stimulation element configured to provide supplementary therapeutic effects in conjunction with phototherapy. The stimulation element may be selected from, or include combinations of, an electrical stimulation module, a thermal stimulation module, or a mechanical stimulation module. For example, the electrical stimulation module may be disposed adjacent to or integrated with the light guide housing, and may deliver low-level microcurrent or neuromuscular stimulation through conductive contact surfaces arranged along the applicator portion of the device. Such stimulation assists in promoting localized blood circulation, enhancing muscle relaxation, and supporting tissue regeneration within the pelvic region.

In other embodiments, the stimulation element may comprise a thermal regulation unit, such as a resistive heater or Peltier-based module, that provides mild heating or cooling cycles. This feature can be synchronized with the light therapy cycle, thereby amplifying therapeutic outcomes by soothing discomfort, reducing inflammation, or improving user comfort during treatment.

Additionally, the stimulation element can be operatively controlled through the same user interface described with reference to FIGS. 1 to 3. For instance, a third button may be configured to switch the stimulation modes between off, low, medium, and high intensity. Alternatively, the stimulation element can be automatically activated in pre-programmed cycles coordinated with the illumination settings to ensure optimal therapy.

In an embodiment, the phototherapy device may integrate thermal modulation, additional light wavelengths, pulsed or patterned therapy modes, Bluetooth/Wi-Fi connectivity, visual or auditory feedback, and ergonomic improvements such as antibacterial coatings or adjustable curvature, which can be incorporated without altering the primary structure of the handle 102 or treatment section 104.

In an embodiment of the present invention, a method for providing therapy to a pelvic cavity of a female subject is provided. The pelvic therapy device is configured to deliver a combination of phototherapy and stimulation therapy within the pelvic cavity of a female subject. A typical method of operation proceeds as follows.

First, the subject inserts the applicator portion of the device into the pelvic cavity, ensuring that the light-emitting region and the stimulation surfaces of the applicator are in direct contact with, or in close proximity to, the pelvic tissue. The applicator portion includes a plurality of light-emitting diodes (LEDs) arranged in multiple strips and positioned both axially and radially to provide uniform light irradiation.

Once positioned, the user activates the device by pressing the power button, thereby enabling the light-emitting elements. Upon activation, the system defaults to Mode M1, wherein red light (630 nm) and near-infrared light (830 nm) are simultaneously emitted. These wavelengths fall within the 600-1100 nm therapeutic window, and are capable of penetrating pelvic tissue to promote improved blood circulation, enhanced cellular metabolism, and stimulation of collagen synthesis.

During therapy, the user may adjust brightness levels between 100% and 50% to control irradiation intensity according to comfort or clinical requirement. The light mode button allows cycling through additional therapeutic light regimens, including blue light at 415 nm (antibacterial, surface-level treatment) or red light alone at 630 nm (shallow penetration, tissue repair). This selective wavelength configuration ensures treatment versatility depending on the subject's condition.

In parallel, the stimulation element may be actuated using the dedicated vibration button. The vibration actuator initially operates at 10 Hz and can be cycled through higher frequencies of 50 Hz, 90 Hz, and 120 Hz, each frequency corresponding to progressively stronger mechanical stimulation. This stimulation promotes pelvic floor muscle contraction, neuromuscular re-education, and improved tissue responsiveness.

The device is further configured to operate in combined modes: In Mode M1, therapeutic light is emitted without vibration; and in Mode M2, therapeutic light is emitted in combination with vibration.

In an embodiment, the device may be programmed to deliver alternating cycles of light therapy and vibration therapy, thereby avoiding overstimulation while ensuring cumulative therapeutic benefit.

Each therapy session is preprogrammed to last approximately 10 minutes, a duration selected to balance treatment efficacy with user safety. This time frame ensures sufficient delivery of therapeutic light energy while preventing thermal buildup, as the staggered bead arrangement and radial distribution of the LEDs eliminate localized hot spots.

In some embodiments, the stimulation module may alternatively comprise a thermal element, such as a resistive heater or thermoelectric cooler, capable of adjusting pelvic tissue temperature by 2-5° C. This thermal modulation enhances vasodilation, reduces muscular tension, and complements the effects of phototherapy.

Accordingly, the method of use combines light irradiation and stimulation therapy either concurrently or sequentially, enabling a synergistic effect on pelvic health and addressing a range of therapeutic applications, including pelvic floor rehabilitation, improved circulation, reduction of muscle spasm, and relief of discomfort.

The phototherapy device of the present invention is particularly suited for gynecological and pelvic health applications. The device may be used to deliver therapeutic light energy for alleviating pelvic pain, promoting post-partum tissue recovery, treating vaginal atrophy, and supporting wound healing after surgical or obstetric procedures. In urogynecology, the device may aid in reducing inflammation, stimulating circulation, and improving tissue elasticity in cases of urinary incontinence or pelvic floor dysfunction. The compact design allows both professional clinical use and home use under medical supervision, offering women a non-invasive and user-friendly option for managing pelvic health conditions.

The invention provides multiple advantages over conventional pelvic treatment devices. The integration of phototherapy with optional vibration, micro-current stimulation, and controlled heat or cooling offers a multimodal therapeutic approach, enhancing treatment outcomes and patient comfort. The ergonomic housing, sealing structures, and smooth treatment surfaces ensure safe insertion, minimize the risk of infection, and allow easy cleaning. The modular assembly simplifies replacement and sterilization, while the waterproof sealing provides durability and long-term reliability. Furthermore, uniform light distribution through the light guide housing ensures consistent therapeutic exposure to targeted tissues, improving efficacy while reducing treatment time.

In summary, the device combines safety, comfort, and multifunctionality in a compact form factor, addressing long-standing challenges in women's pelvic therapy. By enabling non-invasive, multi-modal treatment in clinical and personal care settings, the invention enhances patient compliance, improves therapeutic outcomes, and expands accessibility to advanced pelvic health solutions.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A pelvic therapy device comprising:
a housing having an applicator portion configured for insertion into a pelvic cavity of a female user, and a handle, the applicator portion being disposed at a predetermined angle relative to the handle;
a light therapy module disposed within the applicator portion, the light therapy module comprising a light guide housing having an annular cross-section and a plurality of light strips extending axially along the light guide housing, each light strip comprising a plurality of light-emitting beads; and
a light-transmissive member disposed at the applicator portion for directing the therapeutic light toward pelvic tissue; and
a stimulation element disposed within the applicator portion and configured to deliver at least one of electrical, thermal, or mechanical stimulation to pelvic tissue.

2. The pelvic therapy device of claim 1, wherein the predetermined angle between the applicator portion and the handle is in the range of about 140° to 170°, preferably about 158°.

3. The pelvic therapy device of claim 1, wherein the applicator portion includes a distal tip having three light-emitting beads distributed circumferentially at approximately 60° intervals relative to a central axis of the applicator portion, the beads being configured to provide therapeutic irradiation to surrounding pelvic tissue.

4. The pelvic therapy device of claim 1, wherein the plurality of light strips are disposed circumferentially within a single light guide housing to provide expanded irradiation area and uniform coverage of pelvic tissue.

5. The pelvic therapy device of claim 1, wherein the plurality of light-emitting beads are arranged in both axial and radial directions relative to the light guide housing to achieve uniform distribution of therapeutic light and reduce localized heating.

6. The pelvic therapy device of claim 1, wherein the light-emitting beads on adjacent light strips are arranged in a staggered configuration to prevent localized heat accumulation and improve homogeneity of irradiation.

7. The pelvic therapy device of claim 1, wherein the applicator portion further comprises a bracket extending axially within the light guide housing, the light strips being mounted on the bracket.

8. The pelvic therapy device of claim 7, wherein the bracket comprises a support arm connected to the handle and a sleeve covering the support arm, the light strips being clamped between the support arm and the sleeve.

9. The pelvic therapy device of claim 8, wherein the sleeve defines a plurality of openings aligned with the light-emitting beads to permit radial emission of therapeutic light.

10. The pelvic therapy device of claim 8, wherein the support arm includes opposed mounting surfaces having grooves, and the sleeve includes inward protrusions with protruding strips that engage the grooves to secure the light strips.

11. The pelvic therapy device of claim 1, wherein the light guide housing comprises a first protruding ring on its outer peripheral wall, and the housing includes a second protruding ring on an inner wall, the first and second protruding rings being in sealing engagement to provide fluid resistance.

12. The pelvic therapy device of claim 1, wherein the stimulation element comprises a vibration actuator disposed within the applicator portion and configured to generate vibration frequencies selectable between 10 Hz, 50 Hz, 90 Hz, and 120 Hz.

13. A pelvic therapy device, comprising:
a handle configured for grasping by a user;
an applicator portion extending from the handle at a predetermined angle, the applicator portion including a distal tip and a light-transmissive housing;
a light therapy module disposed within the applicator portion, the light therapy module comprising a plurality of light-emitting beads arranged axially and radially along the applicator portion, the distal tip including at least three light-emitting beads distributed circumferentially to provide substantially uniform irradiation of surrounding pelvic tissue;
and a stimulation element disposed within the applicator portion and configured to deliver at least one of electrical, thermal, or mechanical stimulation to pelvic tissue, wherein the angled configuration of the applicator portion relative to the handle and the arrangement of the distal tip light-emitting beads are configured to provide improved contact and therapeutic coverage of the pelvic cavity.

14. The pelvic therapy device of claim 13, wherein the predetermined angle between the applicator portion and the handle is in the range of about 140° to 170°, preferably about 158°.

15. The pelvic therapy device of claim 13, wherein the distal tip light-emitting beads are positioned at approximately 60° intervals relative to a central axis of the applicator portion.

16. The pelvic therapy device of claim 13, wherein the applicator portion further comprises a light-transmissive distal window configured to direct light emitted from the distal tip light-emitting beads toward a forward-facing portion of the pelvic tissue, thereby providing comprehensive irradiation coverage along both the axial and radial directions of the pelvic cavity.

17. A method of providing pelvic therapy to a female subject, the method comprising:
providing a pelvic therapy device comprising a housing having an applicator portion and a handle, the applicator portion being disposed at a predetermined angle relative to the handle;
inserting the applicator portion into a pelvic cavity of the subject, the applicator portion comprising a plurality of light-emitting elements and a stimulation element;
emitting therapeutic light from the light-emitting elements through a light-transmissive portion of the applicator portion to irradiate pelvic tissue; and
actuating the stimulation element to deliver at least one of electrical, thermal, or mechanical stimulation to the pelvic tissue concurrently with or sequentially to the therapeutic light.

18. The method of claim 17, wherein emitting therapeutic light comprises irradiating pelvic tissue with light of a wavelength between about 600 nm and 1100 nm, including at least one of 630 nm red light, 830 nm near-infrared light, and 415 nm blue light.

19. The method of claim 17, wherein actuating the stimulation element comprises providing thermal stimulation to raise or lower temperature of the pelvic tissue by at least 2-5° C.

20. The method of claim 17, wherein the therapeutic light and the stimulation element are operated in alternating cycles of light emission and stimulation delivery.
